(19) United States
(12) Patent Application Publication
(10) Pub. No.: US 2015/0105719 A1
(43) Pub. Date: Apr. 16, 2015

(54) CANNULA COMPRISING A STYLET UNIT ARRANGED IN A DISPACEABLE MANNER

(71) Applicant: Hans Haindl, Wennigsen (DE)

(72) Inventor: Hans Haindl, Wennigsen (DE)

(21) Appl. No.: 14/399,665

(22) PCT Filed: Apr. 30, 2013
(86) PCT No.: PCT/EP2013/059031

§ 371 Date: Nov. 7, 2014

(30) Foreign Application Priority Data
May 9, 2012 (DE) 10 2012 104 058.3

(51) Int. Cl. A61B 17/34

(52) U.S. Cl. CPC A61B 17/3403; A61B 17/3421; A61B 17/3468; A61B 2017/3433; A61B 2017/347
USPC 604/60; 604/165.02

(57) ABSTRACT

This application relates to a cannula (78) comprising a bevelled cannula tip (80) at the distal end and a cannula holder (16) holding the cannula (78) at the proximal end, as well as a stylet unit (82), which can be displaced from a starting position to an end position in the cannula (78), wherein the cannula (78) is particularly intended to deposit at least one element (18, 20) in a body. According to the invention, in order to ensure simple and reliable protection of the cannula tip as well as a locking of the stylet unit in the end position, the distal end (84) of the stylet unit protrudes a distance (A) beyond the cannula tip (80) in the end position and the stylet unit (82) is fixed in the end position while simultaneously covering the bevelled cannula tip (80).

The application relates to a cannula comprising a bevelled cannula tip and a cannula holder holding the cannula at the proximal end, as well as a stylet unit that can be displaced from a starting position to an end position in the cannula, wherein the distal end of said stylet unit protrudes a distance beyond the cannula tip in the end position, and wherein the stylet unit is fixed in the end position while simultaneously covering the bevelled cannula tip.

A cannula of the type mentioned at the outset is known from the WO-A-01/68168. In addition to a holder that is connected with the cannula, a sleeve-shaped protective element is provided which can be displaced in the longitudinal direction relative to the cannula and starts out from the stylet unit, in order to deposit an element that exists in the cannula. The protective element covers the cannula tip, wherein the stylet unit in addition protrudes beyond the tip. The stylet unit itself starts out from the sleeve-shaped protective element.

A cannula can be found in EP 0 574 434 B1, in which the cannula tip after use is covered with a protective cap which is preferably in the form of a hollow cylinder that is open on one end. An element can be deposited in a body by means of an appropriate cannula.

The purpose of the present invention amongst other things is to develop a cannula for depositing an element of the type mentioned at the outset such that simple and reliable protection of the cannula tip as well as locking the stylet unit after using the cannula is ensured, particularly after depositing an element.

According to a further aspect, it is to be accomplished that when at least one element is to be deposited by means of the cannula, it is possible that the element can be arranged in the cannula, and/or in an applicator comprising a cannula, so that it cannot drop out.

A further aspect is to facilitate that following the discharge of a first element, a second element will remain securely within the cannula and not be released unintentionally.

One aspect finally aims to ensure that the stylet unit is securely fixed in its starting position.

At least one of the aspects, in particular the one which is to provide the simple and reliable protection of the cannula tip as well as protecting against damage following the use of the cannula, is essentially solved in that the stylet unit is fixed in position by interaction with the ground cannula tip or by latching in the cannula holder.

The fixing in position cannot be detached non-destructively, i.e. during normal use it is non-detachable.

If the stylet unit is fixed in position by interaction with the ground cannula tip, it can penetrate into the stylet unit, in particular when the stylet unit is retracted from its end position.

A further preferred embodiment is characterized in that the cross-sectional expansion of the stylet unit on its distal end is such that when the stylet unit is laid against the inner surface of the ground cannula tip, a peripheral edge extends radially to the outside over the cannula tip.

According to a further embodiment, it is provided that the cannula tip comprises an outside diameter that is smaller than the inside diameter of the cannula, that the cross-sectional expansion comprises an outside diameter that corresponds to the inside diameter of the cannula, wherein the distal end of the stylet unit comprises a cylindrical surface, i.e. a cylindrical geometry, and wherein the cannula tip in the direction of the longitudinal axis of the cannula is curved inward such that the cannula tip attaches fixed in position at the transition of the peripheral edge that is formed by the sections having different diameters, and which limits the cross-sectional expansion as well as the cylindrical surface, while simultaneously covering the ground cannula tip.

A further embodiment is characterized in that the distal end of the stylet unit comprises a conical surface, i.e. a conical geometry, wherein the conical surface extends from a first diameter which essentially corresponds to an internal diameter of the cannula to a second diameter which is smaller than the inside diameter, wherein the cannula tip has a projection in the area of a rear ground bevel that extends in direction of the longitudinal axis, and said projection bears against the conical surface and presses same against the inner surface of the cannula tip, wherein the cannula tip is fixed in position bearing against the peripheral edge of the cross-sectional expansion.

In particular it is provided that the section of the cross-sectional expansion corresponds to a circle segment, from the chord of which the cylindrical and/or conical section of the stylet unit starts out.

The cannula tip can comprise a relief facet to improve the fixation.

According to a further preferred embodiment, it is provided that the stylet unit comprises a latching element such as locking hooks, which can be locked non-detachably into an undercut that is molded into the cannula holder and/or into a recess of the cannula holder or an equally acting element in the end position of the stylet unit.

According to a preferred embodiment it is provided that the end of the stylet unit protrudes beyond the cannula tip at a distance (A), wherein the distance (A) is in the range between 1 mm≦A≦5 mm, preferably A=2 mm.

Also an end section of the stylet unit can be configured conically, wherein the conical section of a diameter, which essentially corresponds to the inside diameter of the cannula, extends to a diameter that is smaller than the inside diameter, and wherein the stylet unit comprises a distal end the outside diameter of which corresponds to the inside diameter of the cannula.

It is furthermore provided that the cannula tip in the area of the rear ground bevel comprises an inward facing molded shape in the direction of a longitudinal axis of the cannula, which attaches in the end position of the stylet unit against the surface of the end section, which has a conical shape, so that the end section bears against the inside wall of the cannula.

In this embodiment, the cannula tip can comprise a relief facet.

Further particulars, advantages and features of the invention can be derived not only from the Claims, the features to be derived from these, either on their own and/or in combination, but also from the following description of the preferred embodiments to be derived from the drawing;

the drawings show:

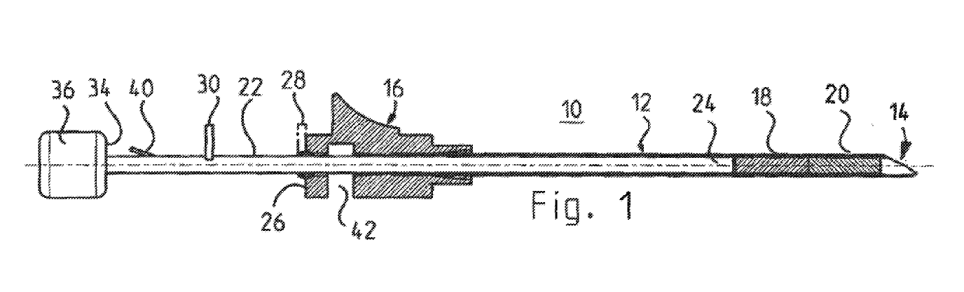

FIG. 1 is a cross-sectional view of a first embodiment of an applicator with an element accommodated in a cannula and a displaceable stylet unit in the cannula,

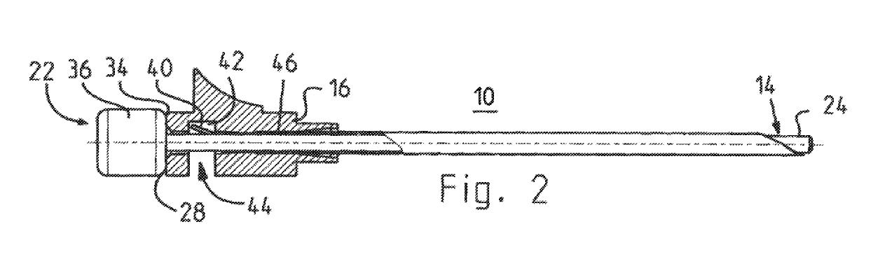

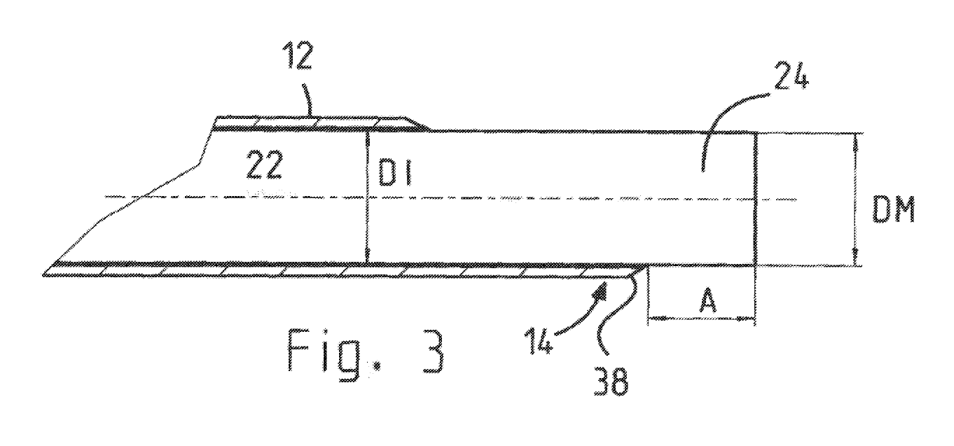

FIG. 2 is a cross-sectional view of the applicator according to FIG. 1, wherein the cannula on the stylet unit is completely retracted,

FIG. 3 is a cross-sectional view of a first embodiment of a stylet unit/end/cannula tip arrangement,

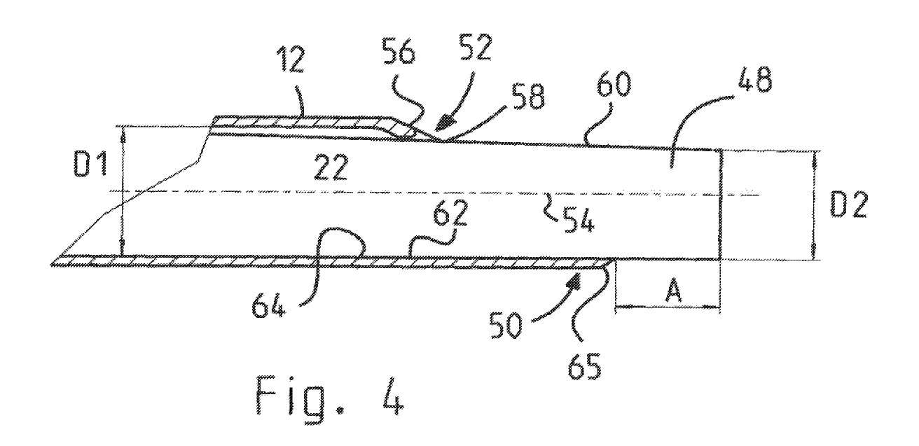

FIG. 4 is a cross-sectional view of a second embodiment of a stylet unit/end/cannula tip arrangement,

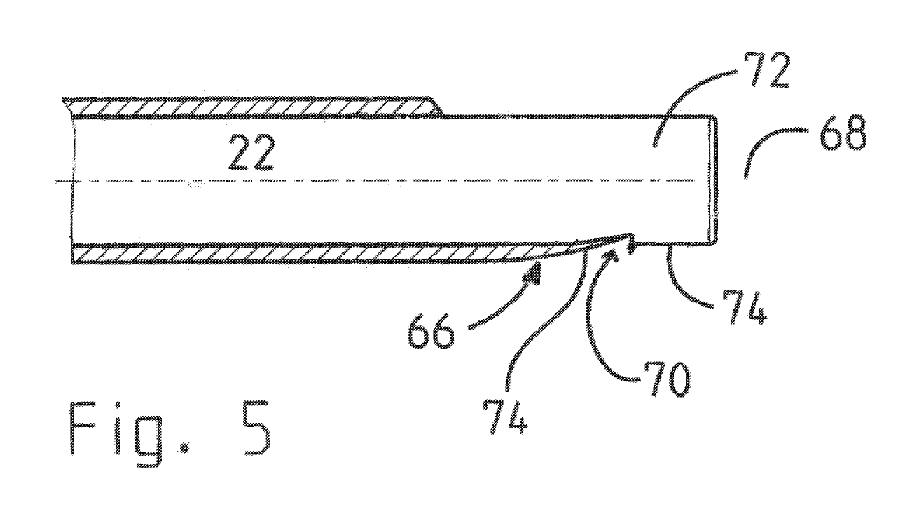

FIG. 5 is a cross-sectional view of a third embodiment of a latching between a stylet unit tip and a cannula tip,

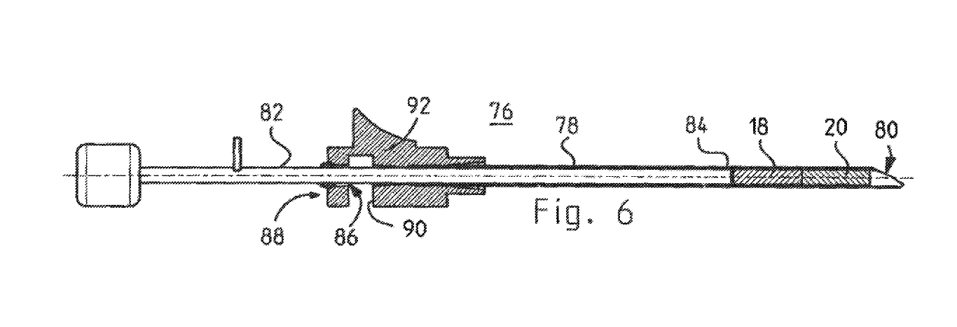

FIG. 6 is a cross-sectional view of a second embodiment of an applicator with a cannula with implants and a displaceable stylet unit in the starting position,

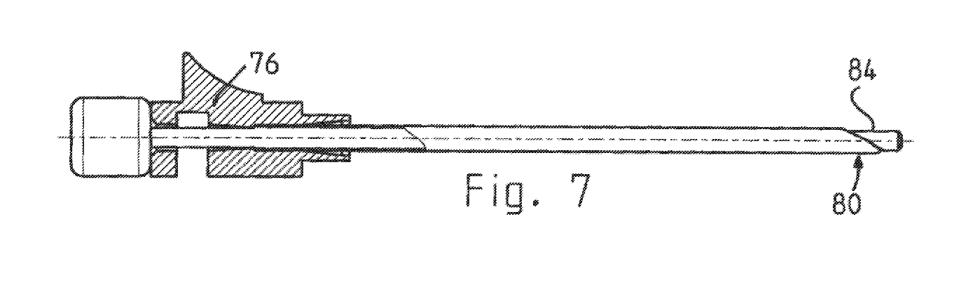

FIG. 7 is an applicator according to FIG. 6 with the cannula completely retracted,

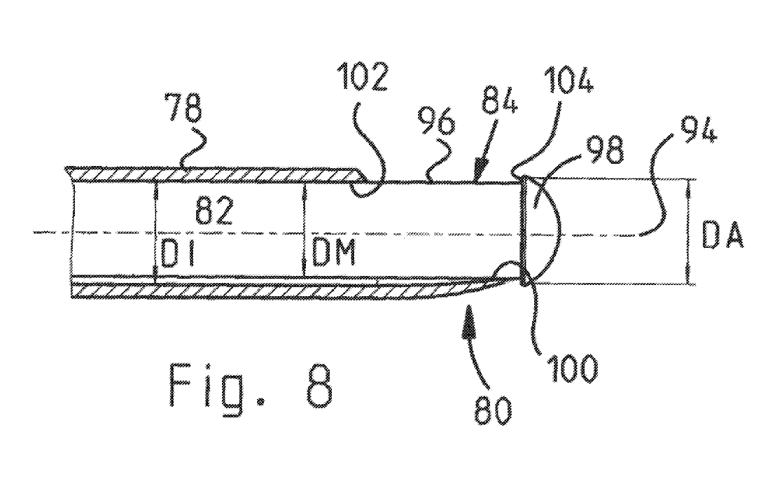

FIG. 8 is a fourth embodiment of a stylet unit/end/cannula tip arrangement,

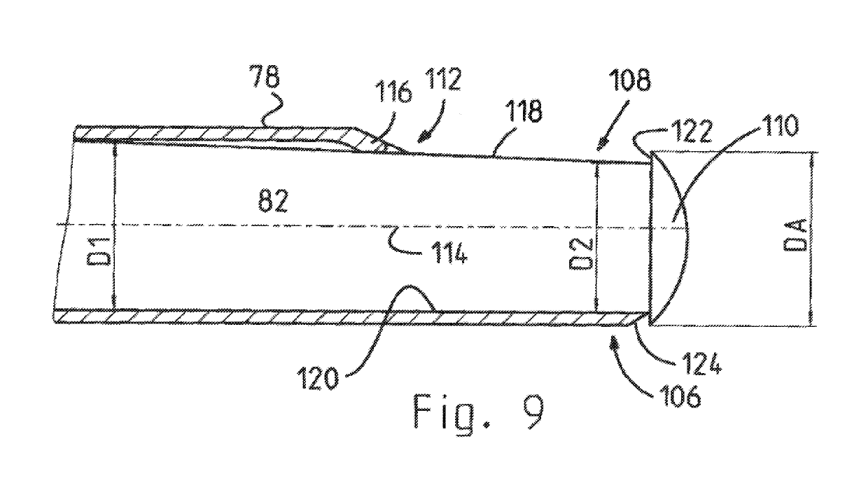

FIG. 9 is a fifth embodiment of a stylet unit/end/cannula tip arrangement,

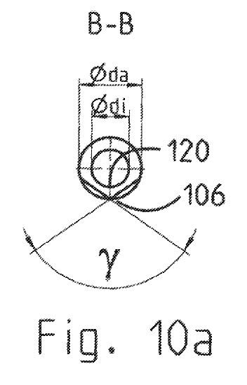

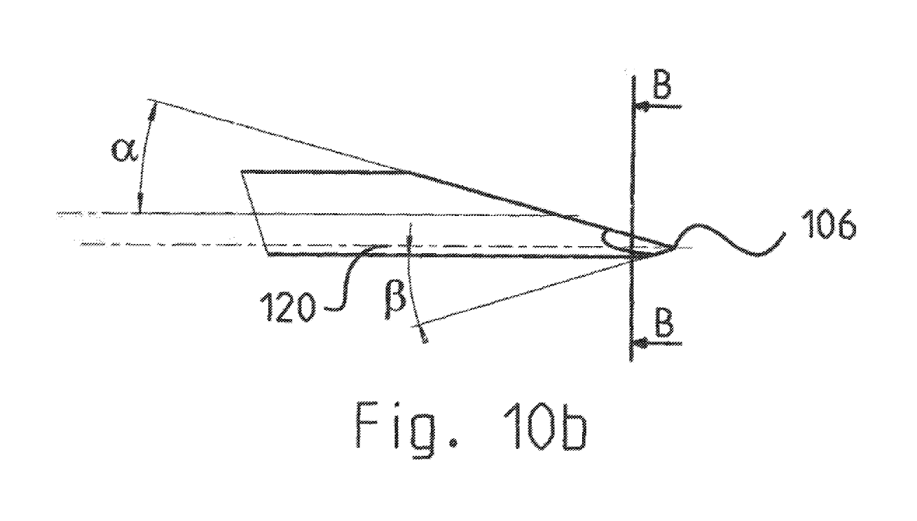

FIG. 10a, 10b is a side elevation of the cannula tip with a faceted cut as well as a sectional representation of a cross-section of the cannula tip.

In the following, the invention is explained using the example of an applicator, which comprises a cannula with a stylet unit that is displaceable within, in order to deposit an element such as an implant in the body of an organism, without limiting the invention hereby. The inventive teaching is rather applicable generally for a cannula with a stylet unit that is displaceable in the longitudinal direction.

FIG. 1 illustrates a sectional representation of a first embodiment of an applicator 10, comprising a cannula 12 with a beveled tip 14 on its front, i.e. distal end, and a cannula holder 16 holding the cannula 12 on its rear, i.e. proximal end. The applicator 10 can be filled with two elements such as implants 18, 20, or be loaded with implants manually. The implants 18, 20 are fixed in the cannula 12 so that they cannot drop out and can be deposited in a tissue (not shown) by means of a stylet unit 22 that is displaceably arranged in the cannula 12.

In the unused condition represented in FIG. 1, the cannula 12 is in a defined starting position, wherein one end 24 of the stylet unit 22 bears against the second implant 18. In the starting position, the cannula 12 bears against a front face 26 of the cannula holder 16 against a first projection like stop 28, which extends vertically relative to the longitudinal extension of the stylet unit. As a result, the starting position is defined; in this position it is prevented that the implants 18, 20 are accidentally ejected.

A second stop 30 is arranged at a distance that corresponds approximately to a length of the first implant 20. The second stop 30 creates a stop as soon as the first implant 20 is released. The stops 28, 30 are arranged such that these can be easily broken off with one finger, for example. For this purpose, they will preferably have a predetermined fracture point.

FIG. 2 shows the applicator 10 in a used condition, wherein the implants 18, 20 are not inserted. In this condition, one front face 34 of an actuator 36 of the stylet unit 22 bears against the front face 26 of the cannula holder 16. In this condition, the end 24 of the stylet unit 22 projects beyond the cannula tip 14. The applicator 10 is characterized in that the stylet unit 22 is locked in the cannula 12 non-detachably after the second applicator 18 is discharged, and the cannula tip 14 is securely protected at the same time.

FIG. 3 illustrates an enlarged representation of the protruding end 24 of the stylet unit 22 which protrudes from the cannula tip 14 at a distance A, as a result of which the tip 14 is covered. In this embodiment, the end 24 of the stylet unit 22 is essentially cylindrical and comprises a diameter DM which essentially corresponds to the inside diameter DI of the cannula 12. In this embodiment, the cannula tip 14 can alternatively comprise an internal facet cut, or, as shown in FIG. 3, can comprise a relief facet 38.

The end 24 of the stylet unit 22 that projects beyond the tip 14 at a distance A covers the tip 14 of the cannula 12 reliably, thus excluding any risk of injury to a user. A non-detachable fixing of the stylet unit 22 in the cannula 12 is accomplished by a latching element 40 in the form of a locking hook, for example, which protrudes from the stylet unit 22, and, when the stylet unit 22 in the cannula 12 reaches a position where it bears against the actuator 36 on the cannula holder 16, it latches into a recess such as an undercut 42 that has been introduced into the cannula holder 16. The recess and/or the undercut 42 can be developed as an opening 44 in the cannula holder 16 that runs transverse to the longitudinal extension of an aperture 46 that accommodates the stylet unit 22.

As soon as the cannula holder 16 bears with its front face 26 against the actuator 36, the locking hook 40 expands into the undercut 42 and in this way ensures a non-detachable locking of the stylet unit 22 in the cannula 12.

FIG. 4 shows a further embodiment of one end 48 of the stylet unit 22 which is conical, and which tapers from a diameter D1, which essentially corresponds to the inside diameter DI of the cannula, to a diameter D2. In this embodiment too, the end 48 of the stylet unit 22 extends beyond the distal end, i.e. the tip 50, beyond the cannula 12, at a distance A.

In this embodiment it is provided that the tip 50 of the cannula 12 in the area of the rear ground bevel 52 comprises a projection 56 which extends in the direction of the longitudinal axis 54 of the cannula 12, said projection with its end section 58 facing the surface area 60 bears against the conical end section 458 and presses against same, so that a force is applied on the opposite surface area 62 of the conical end 48 against the inside wall 64 of the cannula. In this embodiment, the cannula tip 50 preferably comprises a relief facet 65, which engages into the surface 62 of the conical end 48 for locking.

For reliable locking of the stylet unit 22, in this embodiment the stylet unit 22 can also be developed with a locking hook 40, which engages into the recess as well as in the undercut 42 of the cannula holder 16.

FIG. 5 illustrates a third embodiment of a cannula tip 66, which is curved inward in the direction of a longitudinal axis 68 of the cannula 12. In this embodiment, an end 72 of the stylet unit 22 that protrudes beyond the distal end 70 of the tip 66 is fixed, in that the tip 66 presses itself into the surface 74 of the end 72. In this embodiment, the end 72 of the stylet unit 22 is essentially cylindrical and comprises a diameter that essentially corresponds to the inside diameter of the cannula 12. Furthermore, at least the end 72 of the stylet unit 22 should consists of a thermoplastic material such as polypropylene (PP), polystyrene (PS), so that the tip 66 can press itself into the surface 74. Consequently, a non-detachable (that cannot be detached non-destructively) fixing of the stylet unit for protection of the tip 66 exists. Also in this embodiment it is an advantage, if the tip 66 has a relief facet 74, even though such type of embodiment can also be realized with a traditional tip that has a facet on the inside.

FIG. 6-FIG. 9 illustrate embodiments, in which it is ensured that the cannula tip is protected and the stylet unit is fixed within the cannula exclusively by the design of the cannula tip in conjunction with the design of the end of the stylet unit.

FIG. 6 illustrates a sectional representation of an applicator 76 comprising a cannula 78 with tip 80 and with elements 18, 20 to be implanted. A stylet unit 82 is arranged displaceably in the cannula 78, comprising a distal end 84 that bears against the rear element 18 in the illustrated starting position.

To prevent uncontrolled displacement of the stylet unit 82 from the cannula 78, the stylet unit 82 comprises a localized cross-sectional expansion 88, such as a peripheral rib. In the starting position represented in FIG. 6, the cross-sectional expansion 88 bears non-positively against a wall of a port or guide 86 holding the stylet unit 82 of a cannula holder 90. The guide 86 terminates in a recess such as in an undercut 92. Thereafter, the cross-section of the guide 86 expands in the further course to the cannula 78, so that once the resistance that has been formed by the localized cross-sectional expansion 86 has been overcome, the stylet unit 82 is freely displaceable.

FIG. 7 illustrates a section of a presentation of the applicator 76 in its end position, in which the stylet unit 82 with its end 84 is locked with the cannula tip 80.

FIG. 8 illustrates an enlarged embodiment of the tip 80. This is curved inwards in the direction of the longitudinal axis 92 of the cannula 78, so that said tip bears against a cylindrical surface 96 of the end 84 of the stylet unit 82. In this embodiment, the end 84 of the stylet unit 82 supplementary preferably comprises a mushroom-shaped cross-sectional expansion 98, which has an outside diameter DA that corresponds to the inside diameter DI of the cannula.

In this embodiment, the stylet unit 82 outside of the cross-sectional expansion 98 comprises a diameter DM, which is smaller than the inside diameter DI of the cannula 78. Furthermore, the clearance between a surface 100 of the cannula tip 80 and the opposite inside wall 102 of the rear ground bevel that bears against a surface 96 of the end 84 of the stylet unit 82 corresponds to the diameter DM of the stylet unit 82, so that the cannula tip 80 during the extraction of the stylet unit 82 strikes against a peripheral edge 104 between the peripheral cylindrical surface 96 and the mushroom-shaped cross-sectional expansion 98, so that the stylet unit 82 is locked, thereby achieving protection of the cannula tip 80.

FIG. 9 illustrates an embodiment of the cannula 78 with a straight cannula tip 106 which therefore runs parallel to the longitudinal axis 114, wherein the distal end 108 of the stylet unit 82 is conical, i.e. that it tapers from a diameter D1, which essentially corresponds to the inside diameter DI of the cannula 78, to a diameter D2, so that when it transitions, it has an expanded cross-sectional expansion 110 compared to the diameter D2, which is preferably mushroom-shaped and comprises a maximum diameter DA that corresponds to the inside diameter of the cannula 78.

In this embodiment, in the area of a rear ground bevel 112, an inward curved projection 116 in the area of the longitudinal axis 114 of the cannula 78 is provided, where said projection bears against the conical surface 118 of the distal end 108 of the stylet unit 82, in order to press the stylet unit 82 under force against the inner surface 120 of the cannula tip 106. As a result, the cannula tip 106 bears against the peripheral edge bordering the cross-sectional expansion 110.

In this embodiment, the cannula 106 is preferably provided with a relief 124.

A conceivable embodiment of the relief 38, 66, 124, is illustrated in the FIG. 10a), 10b). For that purpose, the cannula tip 106 lies in a plane that is defined by the inside wall 120 of the cannula 78, so that the cannula tip 106 on its one side bears against the surface of the stylet unit 82 and on the other bears against the peripheral edge 122 and therefore fixes the stylet unit 82.

For this purpose, the angle α is in the range of 8°≦α≦20°, the angle β is in the range of 20°≦β≦60°, and the angle y is in the range of 20°≦y≦120°. To that extent, the figures are self-explanatory. From FIGS. 10a and 10b it can be seen that the front faces of the ground surface have an angle y which from the plane defined by the distal front face of the cannula and the longitudinal axis include an angle α, and the outer surface of the ground surface in a plane, in which the intersection of the front faces and the longitudinal axis runs, and the longitudinal axis includes an angle β.

The applicator 10 is used as follows. The cannula 12 preloaded with the implants 18, 20 is removed from the packaging. The needle protector is removed, if necessary. After preparing the puncture, the cannula is pushed below the skin up to the proximal marking. While holding the stylet unit 22 at the same time, the cannula 12 with the cannula holder 16 is retracted up to the stop 32. The first implant 20 is therefore released. The cannula 12 is subsequently retracted up to the distal marking. Because of the slightly inward curved tip 14 it is ensured that the second implant 18 cannot slip out during the process. Following the retraction, the cannula 12 is advanced once more at an offset angle. The stylet unit 22 cannot shift during the entire process, because stop 30 prevents it. When the cannula 12 is inserted again up to the second marking, the stop 30 is broken off and the cannula 12 is retracted completely against the stylet unit stop 34. There it will lock into position so that it cannot be detached. At the same time, the tip 14 is protected by the specially designed end of the stylet unit.

1. A cannula (12, 78) with a beveled cannula tip (14, 50, 66, 80, 106) on the distal end and a cannula holder (16) on its proximal end holding the cannula (12, 78), as well as a stylet unit (22, 82) which is holding the cannula (12, 78), which can be displaced from a starting position into an end position, whose distal end (24, 48, 72, 84, 108) in the end position protrudes beyond the cannula tip (14, 50, 66, 80, 106) at a distance (A), and wherein the stylet unit (22, 82) is fixed in the end position while simultaneously covering the beveled cannula tip (14, 50, 66, 80, 106), characterized in that the stylet unit (22, 82) is fixed in position by interaction with the ground cannula tip (14, 50, 66, 80, 106) or by locking in the cannula holder (16).

2. The cannula according to claim 1, characterized in that the ground cannula tip (66, 80) can penetrate into the stylet unit (22, 82) for fixing into position.

3. The cannula according to claim 1 or 2, characterized in that the fixing in position is developed as a non-detachable fixation or as a fixation that cannot be detached non-destructively during normal use.

4. The cannula according to at least one of the preceding claims, characterized in that the stylet unit (22, 82) comprises a cross-sectional expansion (98, 110) on its distal end (84, 108) that is bordered by a peripheral edge (104, 122) which extends transverse to the longitudinal axis (114) of the cannula (78) opposite of the adjacent area of the stylet unit, and against which the cannula tip (80, 106) can bear.

5. The cannula according to claim 4, characterized in that the stylet unit (82) outside of the cross-sectional expansion (98, 110) comprises an outside diameter (DM) that is smaller than the inside diameter (DI) of the cannula (78), in that the cross-sectional expansion (98, 110) comprises an outside diameter (DA) which corresponds to the inside diameter (DI) of the cannula (78), in that the distal end (84) of the stylet unit (82) comprises a cylindrical surface and that the cannula tip (80) is curved inward in the direction of the longitudinal axis (114) of the cannula (78) such that the cannula tip (80) bears against the peripheral edge (104) of the cross-sectional expansion (98) as well as against the cylindrical surface (96) fixed in position.

6. The cannula according to at least one of the preceding claims, characterized in that the clearance between the inside surface (100) of the cannula tip (80) that is curved towards the inside and the inner surface (102) of an opposite ground bevel corresponds to the outside diameter (DM) of the stylet unit (82).

7. The cannula according to at least one of the preceding claims, characterized in that the distal end (108) of the stylet unit (82) comprises a conical section (108), wherein the section (108) of a first diameter (D1) which essentially corresponds to the inside diameter (DI) of the cannula (78) extends to a second diameter (D2) which is smaller than the inside diameter (DI), wherein the cannula tip (106) has a projection (116) in the area of a rear ground bevel (112) that extends in the direction of the longitudinal axis (114), and said projection bears against the conical section (118) and presses same against the inner surface (120) of the cannula tip (106), and that the cannula tip (106) is fixed in position bearing against the peripheral edge (122) of the cross-sectional expansion (110).

8. The cannula according to at least one of the preceding claims, characterized in that the section of the cross-sectional expansion (110) corresponds to a circle segment, from the chord of which the cylindrical and/or conical section of the stylet unit (82) starts out.

9. The cannula according to at least one of the preceding claims, characterized in that the distal end (48) of the stylet unit (22) comprises a conical surface (60), wherein the conical surface (60) of a first diameter (D1), which essentially corresponds to the inside diameter (DI) of the cannula (12, 78), extends to a second diameter (D2) that is smaller than the inside diameter (DI).

10. The cannula according to at least claim 9, characterized in that the cannula tip (50) in the area of a rear ground bevel (52) comprises an inward molded shape (56) in the direction of the longitudinal axis (54) of the cannula (12) and said molded shape bears against the conical section of the distal end (48) in the end position of the stylet unit (22), so that the conical surface (60) bears against the inner surface (64) of the cannula tip (50).

11. The cannula according to at least one of the preceding claims, characterized in that the cannula tip (14, 50, 80, 106) comprises a relief facet (38, 65, 124) or that the cannula tip (14, 50, 66, 80, 106) in direction of a longitudinal axis (54, 68, 94, 114) of the cannula (12, 78) is curved inward or that the inward curved cannula tip (66, 80) comprises a relief facet (74).

12. The cannula according to at least one of the preceding claims, characterized in that the distal end (72) of the stylet unit (22) is cylindrical and comprises an outside diameter (DA) that corresponds to the inside diameter (DI) of the cannula (78), and that the cannula tip (66) in direction of the longitudinal axis (68) of the cannula (12) is curved inward and penetrates into the distal end (72) of the stylet unit (22) in the end position.

13. The cannula according to at least claim 12, characterized in that the distal end (72) of the stylet unit (22) comprises thermoplastic material such as polypropylene (PP) or polystyrene (PS) or consists of thermoplastic material.

14. The cannula according to claim 1, characterized in that the stylet unit (22) comprises a latching element (40) such as locking hooks, which can be non-detachably locked into a recess such as undercut (42) that exists on the cannula holder (16) in the end position of the stylet unit (22).

15. The cannula according to at least one of the preceding claims, characterized in that the distal end (24, 48, 72, 84, 108) of the stylet unit (22, 82) protrudes beyond the cannula tip (14, 50, 66, 80, 106) at a distance (A), wherein the distance (A) lies in the range between 1 mm≦A≦5 mm, preferably A=2 mm.

16. The cannula according to at least claim 10, characterized in that in a relief facet (38, 65, 124) of the cannula (12, 78) front faces of the ground surface include an angle γ with 20°≦γ≦120° and/or the outer surface of the ground surface in a plane, in which the intersection of the front faces and the longitudinal axis (54, 68, 94, 114) runs, to the longitudinal axis (54, 68, 94, 114) includes an angle β with 20°≦β≦60° and/or distal front face of the cannula includes an angle α with 8°≦α≦20° to the longitudinal axis (54, 68, 94, 114).

17. The cannula according to at least one of the preceding claims, characterized in that the cannula is intended for depositing at least one element (18, 20) into a body of an organism by interaction with and relative movement between the stylet unit (22, 82) and the cannula (12, 78), wherein the elements (18, 20) prior to the deposition into the body is fixed in position between the stylet unit (22, 82) and the cannula tip (14, 50, 66, 80, 106), or that the at least one element (18, 20) is fixed in position between the distal end (24, 48, 72, 84, 108) of the stylet unit and the cannula tip (14, 50, 66, 80, 106) by means of the inward curved cannula tip (66, 80) or the projection (56, 116) extending in the direction of the longitudinal axis.

18. The cannula according to at least one of the preceding claims, characterized in that the stylet unit (22, 82) comprises a first stop (28), which in the starting position bears against one front face (26) of the cannula holder (16) and/or that the stylet unit (22, 82) comprises a further stop (30), against which the front face (26) of the cannula holder (16) strikes as soon as the first element (20) is released, and/or that the stops (28, 30) are detachably connected with the stylet unit (22), preferably by means of a predetermined fracture point.

19. The cannula according to at least one of the preceding claims, characterized in that the stylet unit (82) in its starting position is fixed non-positively by means of a cross-sectional expansion (88) in a first section of a port (86) of the cannula holder (90) and is freely displaceable in an adjacent section in the direction of the cannula after adjusting the stylet unit.

20. The cannula according to at least one of the preceding claims, characterized in that at least two projections (28, 30) such as stops protrude from the stylet unit (82), which comprise a distance that has at least the equal axial length of an element (18, 20) to be deposited, wherein the projections can in particular be broken off manually.
